(19) United States
(12) Patent Application Publication
(10) Pub. No.: US 2003/0055377 A1
(43) Pub. Date: Mar. 20, 2003

(54) Exchangeable catheter

(75) Inventors: Motasim Sirhan, Sunnyvale, CA (US); Soodabeh Tronson, Redwood City, CA (US); John Yan, Los Gatos, CA (US); Kevin Gertner, Los Gatos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Avantec Vascular Corporation, Sunnyvale, CA

(21) Appl. No.: 10/159,602

(22) Filed: May 30, 2002

Related U.S. Application Data
(63) Continuation-in-part of application No. 10/080,920, filed on Feb. 20, 2002.
(63) Continuation-in-part of application No. 10/001,210, filed on Nov. 30, 2001, now abandoned.
(63) Continuation-in-part of application No. 09/872,640, filed on May 31, 2001.
(63) Continuation-in-part of application No. 09/585,943, filed on Jun. 2, 2000.
(60) Provisional application No. 60/366,079, filed on Mar. 18, 2002.

(51) Int. Cl. A61M 29/00

(52) U.S. Cl. 604/103.04; 600/434

(57) ABSTRACT

An intravascular balloon catheter comprising a tubular catheter shaft having an elongate member receiving lumen extending along a substantial length of the catheter shaft and having an access for directing an elongate member to and from the elongate member receiving lumen.

### CROSS-REFERENCES TO RELATED APPLICATIONS

This application is a continuation-in-part of application Ser. No. 10/080,920 (Attorney Docket No. 020460-000230), filed on Feb. 20, 2002, which was a continuation-in-part of application Ser. No. 10/001,210 (Attorney Docket No. 020460-000220), filed on Nov. 30, 2001, which was a continuation-in-part of application Ser. No. 09/872,640 (Attorney Docket No. 020460-000210), filed on May 31, 2001, which was a continuation-in-part of application Ser. No. 09/585,943, (Attorney Docket No. 020460-000200), filed on Jun. 2, 2000. This application also claims the benefit of provisional application 60/366,079 (Attorney Docket No. 020460-000240), filed on Mar. 18, 2002. The full disclosures of each of these applications are incorporated herein by reference.

### BACKGROUND OF THE INVENTION

1. Field of the Invention

The present invention relates generally to medical devices and methods. More particularly, the present invention relates to a balloon catheter having an exchangeable balloon structure.

2. Description of the Background Art

Percutaneous transluminal angioplasty procedures have become a therapy of choice for treating stenosed regions in the patient's vasculature, particularly the coronary vasculature. Recently, the use of such angioplasty procedures has often been combined with stent placement and/or radiation treatment to inhibit restenosis and hyperplasia following angioplasty. When performing such multiple, sequential treatments, it is usually necessary to “exchange” catheters which are used to perform each of the procedures. That is, the initial angioplasty treatment will be performed using a balloon angioplasty catheter. After the angioplasty is completed, a second catheter carrying a stent or other vascular prosthesis must then be introduced to the treatment site. Introduction of the second catheter involves first removing the balloon angioplasty catheter and then placing the second catheter in the treatment region. Optionally, a third catheter may then be exchanged for the second in order to perform radiation or other treatments in order to inhibit hyperplasia.

In performing such multiple, sequential treatments, most physicians prefer to leave a “guidewire” in place to the treatment location. A guidewire is a small diameter, highly flexible wire that can be steered to the target location through the vasculature and which then acts as a guide path for introducing and positioning the balloon angioplasty and other interventional catheters.

In the early days, balloon angioplasty catheters were designed to be introduced into the vasculature in an “over-the-wire” manner. That is, the catheters were designed to have access, commonly referred to as guidewire lumens, which extended the entire distance from the distal end of the catheter to the proximal end of the catheter. The catheter could then be loaded over a proximal end of a guidewire which was already in place in the patient and then advanced over the guidewire until a distal end of the catheter reached the target site. While functional, the need to maintain control of the guidewire while the interventional catheter was being introduced meant that the guidewire had to have an excess length outside of the patient which was greater than the length of the catheter being introduced. If the length were any shorter, the treating physician would not be able to hold on to the guidewire as the catheter was being introduced. Although necessary for catheter introduction, the excess guidewire length (optionally in the form of a detachable extension) was very difficult to manage during other parts of the treatment.

To overcome the difficulties associated with very long guidewires “rapid exchange” or “monorail” balloon angioplasty catheters were developed. A number of specific designs have been developed over the years, and the rapid exchange catheters generally have a shortened guidewire lumen which extends from a distal tip of the catheter to an exit port located closer to the distal end of the catheter than to the proximal end. By reducing the length of the guidewire lumen, the need for a guidewire having excess length outside of the patient is also reduced.

The use of rapid exchange catheters has become wide spread, and they have proven to be particularly valuable for use as stent delivery catheters. Stent delivery catheters are normally used after an initial angioplasty treatment. In such cases, the angioplasty catheter will be removed and exchanged for the stent delivery catheter. Use of an angioplasty catheter having a rapid exchange design facilitates removal of the angioplasty catheter over short guidewires. Similarly, use of the stent delivery catheter having a rapid exchange design facilitates introduction of the catheter over the guidewire which remains in place in the patient.

Despite their widespread acceptance, rapid exchange catheters suffer from a number of limitations. In particular, the shortened guidewire lumens reduce the “pushability” of the rapid exchange catheters.

The second problem associated with the use of rapid exchange catheters is the inability to exchange or reintroduce a guidewire into the shortened guidewire lumen of the rapid exchange catheter.

For these reasons, it would be desirable to provide improved apparatus, methods, and kits which permit the exchange of catheters and catheter components over shortened guidewires. Particularly, it would be desirable to provide improved balloon angioplasty and other catheters which can be introduced to the vasculature with improved pushability while allowing removal of the catheter over a shortened guidewire and/or which permits exchange of catheter components over the catheter body which remains in place over the guidewire. At least some of these objectives will be met by the invention described in claims herein after.

### BRIEF SUMMARY OF THE INVENTION

The present invention is directed to intracorporeal devices and methods using the same, and more particularly, to intraluminal devices such as catheters including balloon catheters. The devices of the present invention may be used for performing diagnostic and/or treatment applications. The present devices are suitable for use for the treatment of a variety of conditions within different locations of a patient's corporeal body including the patient's vasculature. In particular, the devices can be used in the coronary, peripheral, and cerebral regions of a patient's vasculature for virtually any treatment modality that relies on, but not limited to, balloon expansion, particularly angioplasty, stent placement, and the like.

As used herein, the term “intracorporeal body” refers to body lumens or internal corporeal tissues and organs, within a corporeal body. The body lumen may be any blood vessel in the patient's vasculature, including veins, arteries, aorta, and particularly including coronary and peripheral arteries, as well as previously implanted grafts, shunts, fistulas, and the like. It will be appreciated that the present invention may also be applied to other body lumens, such as the biliary duct, which are subject to excessive neoplastic cell growth. Examples of internal corporeal tissues and organs, include various organs, nerves, glands, ducts, and the like.

In one embodiment, the intravascular catheters of the present invention comprise a treatment or diagnostic structure. In one embodiment the treatment or diagnostic structure is a balloon structure. Without any limitations intended, a balloon catheter will be used to further describe the different features and embodiments of the present invention.

In an embodiment, the device of the invention is an intravascular catheter comprising an elongate catheter shaft having proximal and distal ends, proximal and distal sections, and a diagnostic or treatment structure such as an expandable member such as an inflatable member (e.g., balloon) disposed at the distal section of the elongate shaft and sealingly secured thereto. At least one lumen such as an inflation lumen extends along at least a portion of the catheter shaft. In an embodiment the inflation lumen terminates at a point proximal to the distal end of the catheter shaft, and is in fluid communication with an interior of the balloon. The catheter shaft may be of a unitary construction or formed from more than one longitudinally fluidically connectable portions.

In one embodiment, the catheter shaft further includes an elongate member receiving lumen having proximal and distal ends and extending along at least a portion of the shaft, for slidably and removably receiving or being received over an elongate member such as a tubular body, a guidewire, or a guidewire disposed within a tubular body. In an embodiment the elongate member receiving lumen extends to a distal port at the shaft distal end. The proximal end of the elongate member receiving lumen may be positioned along the length of the catheter shaft, including but not limited to: being at the proximal end of the catheter shaft, being distal to the catheter shaft proximal end and substantially closer to the shaft proximal end than to the shaft distal end; being distal to the catheter shaft proximal end and substantially at equal distance from the shaft proximal and distal ends; being distal to the catheter shaft proximal end and proximal to a balloon proximal end. The multiple lumens of the catheter shaft may be formed a single dual lumen catheter body or from different tubular members having lumens extending therein.

The elongate member receiving lumen includes an access extending along at least a portion of the length thereof, and having proximal and distal ends. At least a portion of the interior of the elongate member receiving lumen is fluidically connectable to outside of the catheter shaft by way of the access. The access may further include longitudinally extending transverse ends. The access transverse ends may overlap, abut, or have an opening formed therebetween. In an embodiment, the transverse ends are sufficiently flexible to allow the insertion and/or withdrawal of the elongate member to and from the elongate member receiving lumen. In an embodiment, the catheter shaft, at least along the transverse ends is formed of a flexible material having elastic characteristics to enable the retraction of the transverse ends back to a retracted state after the insertion and/or withdrawal of the elongate member to and/or from the elongate member receiving lumen. When the transverse ends form an opening therebetween, the opening is sufficiently large to allow the access of the elongate member (e.g., tubular member or guidewire) to and from the elongate member receiving lumen, yet sufficiently small to minimize the unwanted withdrawal of the elongate member from the elongate member lumen.

The access proximal end may be coincident with the proximal end of the elongate member receiving lumen or it may be at a point distal to the elongate member receiving lumen proximal end including but not limited to: being distal to the elongate member receiving lumen proximal end and substantially closer to the shaft proximal end than to the shaft distal end; being distal to the elongate member receiving lumen proximal end and substantially at equal distance from the shaft proximal and distal ends; being distal to the elongate member receiving lumen proximal end and proximal to a balloon proximal end.

The access distal end may be at any point between the access proximal end, and the balloon interior, usually proximal the balloon proximal end.

The access may comprise a single continuous access or intermittent accesses along the length of the elongate member receiving lumen. In one embodiment, the access comprises a continuous access extending from access primal end to the access distal end.

Alternatively, an optional flexible tubular member, such as a sleeve which may be formed integral with the balloon or be present as a separate element, may extend proximally from the balloon along at least a portion of the distal section of the catheter shaft. In an embodiment the sleeve is attached to the catheter shaft.

In an embodiment a hypotube may be extended along at least a portion of the elongate member receiving lumen to further enhance the pushability of the devices of the present invention.

Depending on the intracorporeal target site and the intended clinical use, the dimensional characteristics of devices within the scope of the present invention may vary.

In an embodiment, the elongate member (e.g., guidewire) may be disposed, at least partially, within the elongate member receiving lumen during the procedure. The positioning of the elongate member (e.g., guidewire disposed within a lumen of the tubular member) within the elongate member receiving lumen will provide push strength necessary for improved advancement of the elongate member within the corporeal body. This will also help reduce buckling of the shaft.

In one embodiment, the elongate member is a tubular member including a lumen for receiving another elongate member such as a guidewire therein. The transverse cross section of the catheter may be of any suitable shape including circular, oblong, or elliptical profile.

The distal end of either or both the catheter shaft and the elongate member, preferably, is axially tapered for improved navigation through the tortuous path of the intracorporeal body, usually for a length of at least 3 millimeters (mm), typically at least about 0.5 mm, preferably at least about 0.1 mm. The elongate member, may also include an atraumatic distal tip.

The devices of the present invention may be available independently or as an assembly. Optionally, the assembly may be an aggregate assembly further including at least a second treatment or diagnostic device. By way of example, when available as an assembly, usually, the device such as a balloon catheter will be preloaded over the elongate member (e.g., guidewire, tubular body, guidewire within the tubular body) and the assembly is sterilized and packaged as a complete unit. The second treatment or diagnostic structure, such as a second balloon catheter, may have a elongate member receiving lumen which is slidably receivable over the elongate member. The second balloon catheter may be included as part of a single aggregate assembly together with the first balloon catheter and the elongate member, or it may be available as a separate package for use with the elongate member or the assembly of the present invention.

Typically, the second balloon catheter will differ from the first in someway, such as dimensions, including diameter, length, or both; shape; balloon material; balloon characteristics such as compliance, flexibility, elasticity or the like; or other features. In one embodiment, at least one of the balloon catheters, usually the second balloon catheter may carry a stent or other vascular prosthesis. Usually, but not necessarily, the first balloon catheter is intended for performing angioplasty or other therapeutic or diagnostic procedure with the second balloon catheter being intended to deliver a stent after the angioplasty treatment. Other examples include drug infusion balloons, radioactive delivery balloons, atherectomy, and the like. Of course, the intravascular catheter assemblies comprising only a single balloon catheter may also be adapted to carry a stent, drug infusion balloon, radioactive delivery balloon, or the like, as well.

In an embodiment, the intravascular catheter and/or assemblies of the present invention may further comprise a deployable embolic capture element on either or both the elongate member and the balloon catheter. The deployable embolic capture element may comprise coils, wires, braids, mesh, and the like and take on a variety of shapes such as funnel or parachute shapes. Preferably, the embolic capture element is formed from a nickel-titanium alloy (such as Nitinol™ alloy), spring stainless steel, or like materials and may additionally be coated or contained by a polymer material. The expandable embolic capture element allows for filtering and/or suctioning of any emboli (which may potentially occlude a body lumen) before, during, and/or after treatment with the intravascular catheter. The embolic filter will generally have micro size holes in the range of about 1 micron (um) to about 200 um, usually from about 1 um to about 100 microns for the retrieval of emboli. The embolic filter may be released open and closed, at least in part, by axial or radial movement of the balloon structure or the catheter body.

In another embodiment, the intravascular catheters and/or assemblies of the present invention may further comprise a second expandable balloon on the elongate member configured to be distal to the first balloon catheter. The second balloon may have dimensions, characteristics, and be formed from materials, similar to the first balloon described above. The second balloon may also carry a balloon expandable vascular prosthesis. In some instances, the first balloon may perform angioplasty or other therapeutic or diagnostic procedures while the second balloon may be intended to deliver a stent (balloon expandable) after the angioplasty treatment. Thus, such an embodiment advantageously allows for sequential treatments in a single catheter structure.

In another embodiment, the intravascular catheters of the present invention may comprise a self-expanding vascular prosthesis on the elongate member. The self-expanding prosthesis may be formed from steel, nickel titanium, shape memory alloy, cobalt, composite material, and the like. Typically, the self-expanding prosthesis will be deployed, at least in part, by axial or radial movement of the first balloon catheter and/or the elongate member.

In an exemplary method embodying features of the present invention a guiding catheter is inserted into the coronary artery in a conventional manner. The device of the present invention, such as the balloon catheter is prepared for insertion into the guiding catheter in a conventional manner. An elongate member such as the guidewire is then introduced into the catheter by a back loading technique. The proximal extremity of the guidewire is inserted backwardly through the distal tip at the distal end of the catheter through the elongate member receiving lumen. The guidewire is advanced rearwardly by holding the distal extremity of the balloon catheter in one hand and advancing the guidewire rearwardly. The guidewire may be advanced toward the proximal end of the catheter either within or outside the elongate member receiving lumen. However, the guidewire is preferably maintained within the elongate member receiving lumen during this proximal advancement. The guidewire is advanced proximally until the distal end of the guidewire with its flexible or floppy tip protrudes at least partially from the distal end of the catheter.

The distal end of the catheter with the flexible tip of the guidewire protruding therefrom is then slid down the guiding catheter previously positioned within the patient. The catheter with the guidewire positioned within the elongate member receiving lumen is grasped between the fingers of a hand and is advanced into the guiding catheter. This procedure is continued until a substantial portion of the catheter is disposed in the guiding catheter. With the catheter housing the guidewire in the catheter elongate member receiving lumen during this distal advancement, improved pushability may be achieved.

In one embodiment, the catheter with the guidewire housed within the catheter elongate member receiving lumen is held stable by the fingers of the hand and is advanced distally until the distal end of the guidewire crosses the stenosis which it is desired to be opened or enlarged and the therapeutic or diagnostic portion such as the balloon is at the desired lesion site. Since the guidewire and the catheter are advanced, at least mostly, together, greater pushability can be obtained in advancing the balloon dilatation catheter across the stenosis. In other words, more force can be applied to the balloon to cause it to cross the stenosis or lesion in case the opening therein is very small.

Alternatively, if desired, the proximal extremity of the guidewire may be disposed outside of the catheter elongate member receiving lumen and a torquer can be attached to the guidewire near the proximal end of the guidewire. The guidewire is then advanced ahead of the catheter until it enters the arterial vessel of the patient. The catheter with the guidewire housed within its elongate member receiving lumen is held stable by the fingers of the hand while the guidewire is being advanced distal of the catheter distal end. The positioning of the guidewire in the desired arterial vessel can be observed under a fluoroscope by using x-ray techniques well known to those skilled in the art. As is well known to those skilled in the art, the torquer can be utilized for rotating the guidewire to facilitate positioning of the flexible tip in the desired arterial vessel so that the distal end of the guidewire can be advanced into the stenosis which it is desired to open or enlarge. As soon as the guidewire is in the desired location, with the exposed proximal extremity of the guidewire held stationary by two fingers of the hand, the catheter is advanced over the guidewire until the therapeutic or diagnostic portion such as the balloon is at the desired lesion. If any difficulty is encountered by the operator conducting the procedure in introducing the catheter so that the balloon resists crossing the lesion or stenosis, the guidewire can be retracted slightly. The person then can observe under the fluoroscope to see that the tip of the guidewire is wiggling in the blood stream indicating that it is free to move in the blood stream. Then the operator can grasp both the guidewire and the catheter in one hand and advance them as a unit so that they can cross the stenosis as a unit.

After the balloon has crossed the stenosis or lesion, the balloon can be inflated in a conventional manner by introducing a radiopaque contrast liquid through the catheter inflation lumen. After the inflation has occurred and the desired operation has been performed by enlarging the opening in the stenosis, the catheter can be removed very rapidly by the person performing the procedure by grasping the a proximal extremity of the guidewire outside of the catheter elongate member receiving lumen by two fingers (if a torquer has been used, the torquer may be removed prior to this step).

As the catheter is being retracted proximally out of the guiding catheter, more of the guidewire proximal extremity is removed from the catheter elongate member receiving lumen. If desired, the remainder of the catheter can be removed from the guiding catheter until the distal end of the catheter passes over the proximal end of the guidewire. At this point, if desired, a second catheter (e.g., a second balloon catheter, a second catheter carrying a deployable prosthesis such as a stent) can be loaded onto the guidewire in a rearward direction by introducing the proximal end of the guidewire into the distal tip of the second catheter's elongate member receiving lumen. In an embodiment, the second catheter is a catheter embodying features of the present invention in which event, the second catheter's elongate member receiving lumen may be advanced distally over the guidewire, preferably, with a substantial portion of the guidewire length remaining in the second catheter's elongate member receiving lumen.

The second catheter and the guidewire housed, preferably, substantially within the second catheter's elongate member receiving lumen can then be advanced to the desired location as described before.

It should be appreciated that catheters embodying features of the devices of the present invention may include other types of catheters than balloon dilatation and stent deployment catheters and that these features may be employed in the design and use of other catheters which may require advancement over another elongate member.

### BRIEF DESCRIPTION OF THE DRAWINGS

FIG. 1 is an elevational, partially cutaway, view of a balloon catheter assembly embodying features of the invention.

FIGS. 2P1-5 are transverse cross-sectional views of some of the alternate embodiments of a catheter embodying features of the present invention having a proximal section.

FIG. 3 is an elevational, partially cutaway, view of a balloon catheter embodying features of the invention.

FIGS. 3A1-4 are transverse cross-sectional views of different embodiments of the balloon catheter of FIG. 3 taken along line A-A.

FIGS. 3S1-3 are transverse cross-sectional views of different embodiments of the balloon catheter of FIG. 3 taken along line S-S.

FIG. 3B is a transverse cross-sectional view of an embodiment of the balloon catheter of FIG. 3 taken along line B-B.

FIGS. 3P1-2 are perspective, partially cutaway views, of different embodiments of the catheter of FIG. 3.

FIGS. 4D1-3 are elevational, partially cutaway, views of the alternate embodiments of the distal section of a balloon catheter embodying features of the invention near the proximal extremity thereof.

FIG. 5 is an elevational, partially cutaway, view of an alternate embodiment of the catheter of FIG. 3.

FIGS. 5A1-2 are transverse cross-sectional views of different embodiments of the balloon catheter of FIG. 5 taken along line A-A.

FIGS. 5S1-3 are transverse cross-sectional views of different embodiments of the balloon catheter of FIG. 5 taken along line S-S.

FIG. 5B is a transverse cross-sectional view of an embodiment of the balloon catheter of FIG. 5 taken along line B-B.

FIGS. 5P1-2 are perspective, partially cutaway views, of different embodiments of the catheter of FIG. 5.

FIG. 6 is an elevational, partially cutaway, view of an alternate embodiment of the catheter of FIG. 3.

FIGS. 6A, 6Z, 6S, and 6B, are transverse cross-sectional views of embodiments of the balloon catheter of FIG. 3 taken along lines A-A, Z-Z, S-S, and B-B, respectively.

FIG. 7 is an elevational, partially cutaway, view of an alternate embodiment of the catheter of FIG. 3.

FIGS. 7A, 7N1, and 7N2 are transverse cross-sectional views of different embodiments of the balloon catheter of FIG. 7 taken along lines A-A, N-N, respectively.

FIG. 7P is a perspective, partially cutaway view, of the catheter FIG. 7.

FIG. 8 is an elevational, partially cutaway, view of an alternate embodiment of the catheter of FIG. 3.

FIGS. 8A, 8N, and 8S are transverse cross-sectional views of different embodiments of the balloon catheter of FIG. 7 taken along lines A-A, N-N, and S-S, respectively.

FIGS. 9A1-A5 are transverse cross-sectional views of alternate embodiments of the catheter of FIG. 3 having different access transverse ends.

FIGS. 9T1-3 are top, partially cutaway, views of alternate embodiments of the catheter of FIG. 3.

FIG. 10 is an elevational, partially cutaway, view of an alternate embodiment of the catheter of FIG. 3 including a hypotube disposed therein.

FIGS. 10H, 10N1-3, 10A1-2, and 10S1-2, are transverse cross-sectional views of embodiments of the balloon catheter of FIG. 10 taken along lines H-H, N-N, A-A, and S-S, respectively.

FIGS. 10M1-4 are transverse cross-sectional views of a method of making the catheter of FIG. 10 including a hypotube.

### DETAILED DESCRIPTION OF THE INVENTION

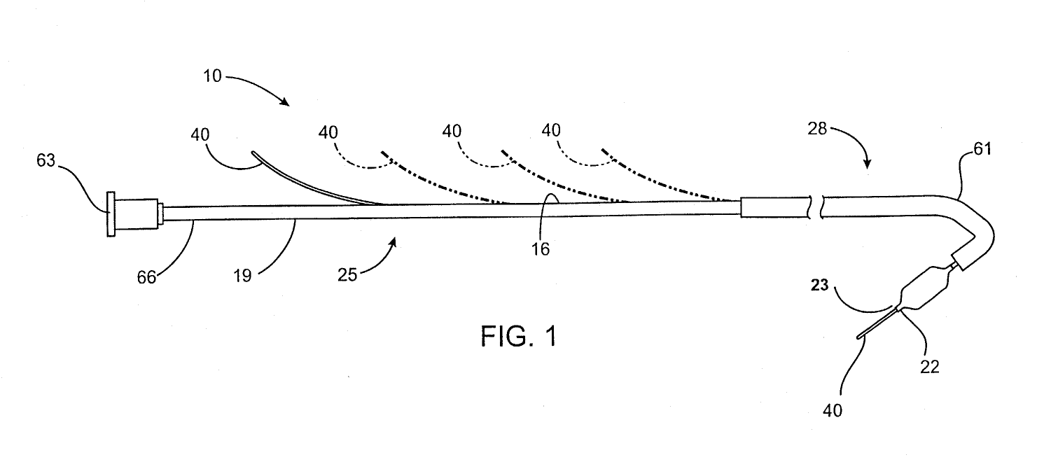

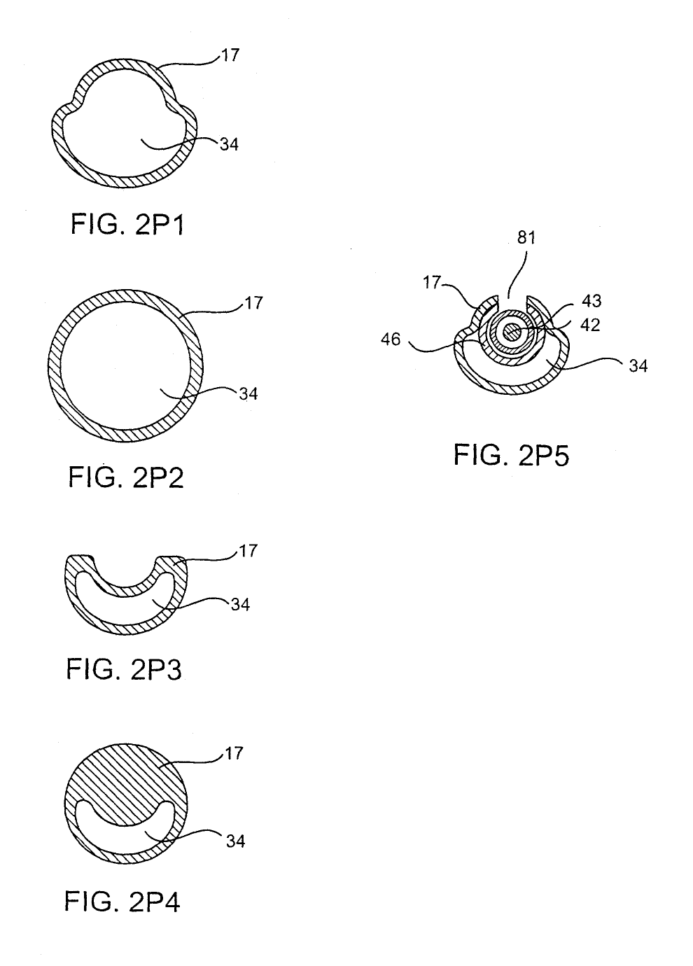

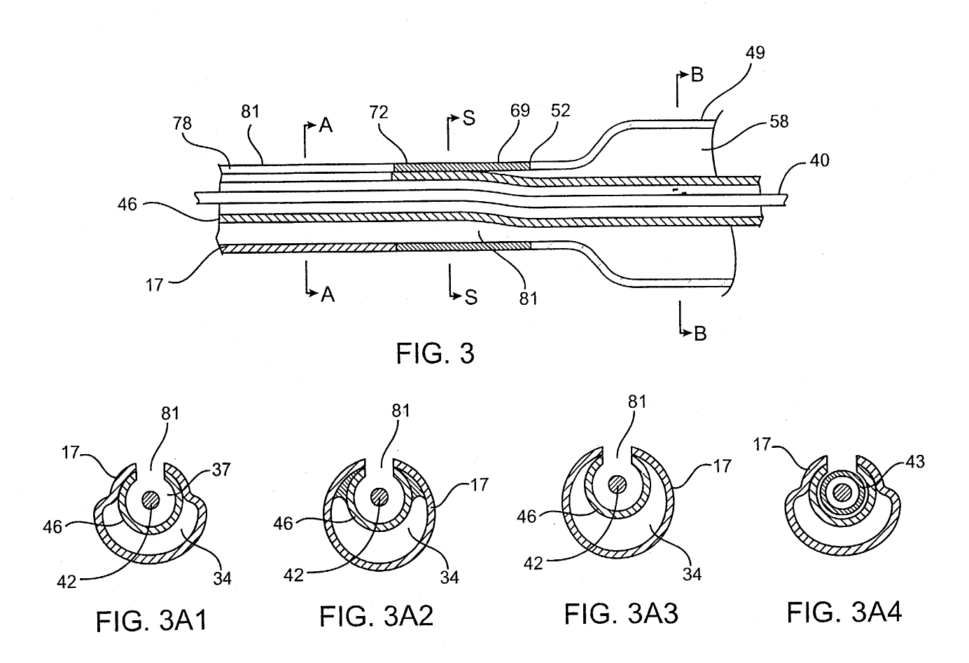

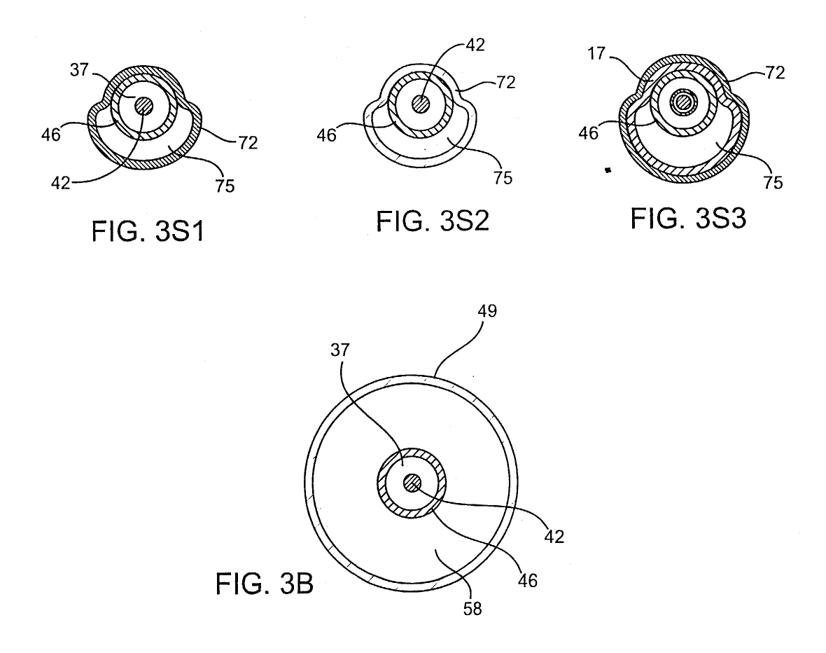

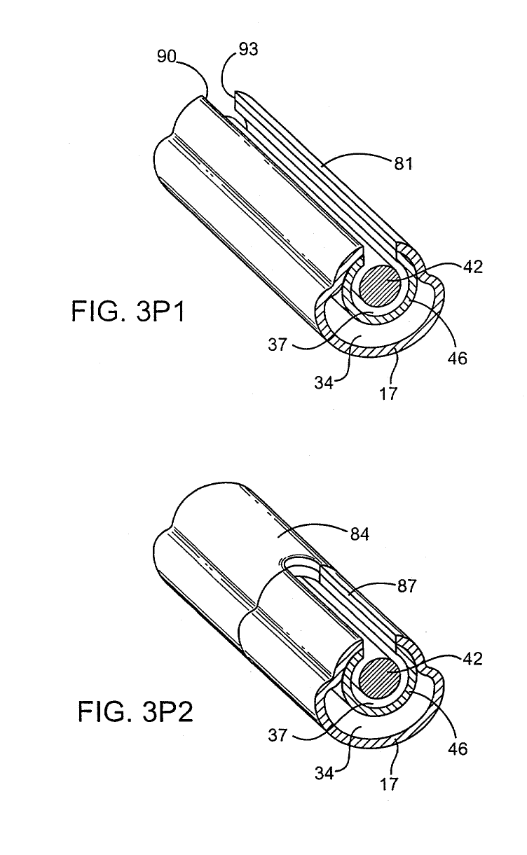

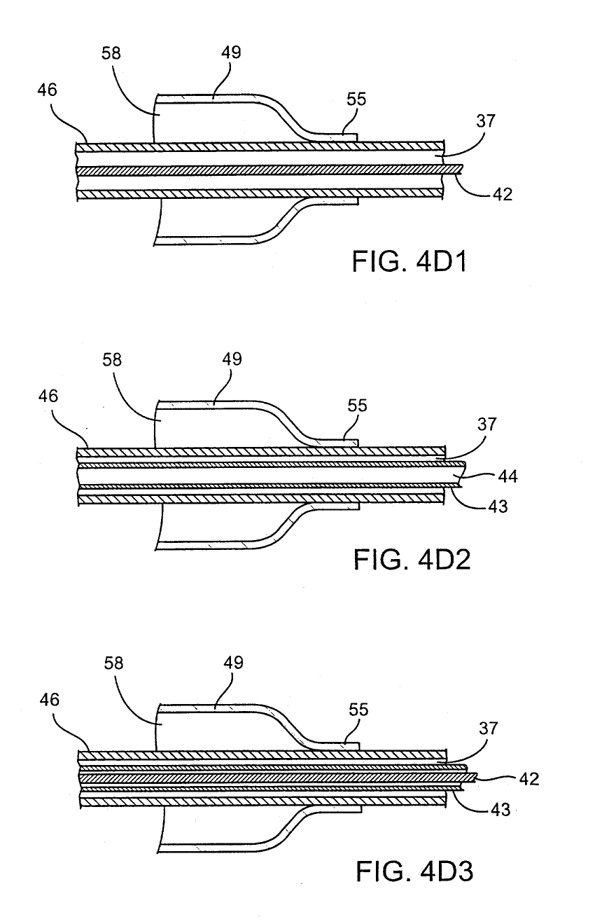

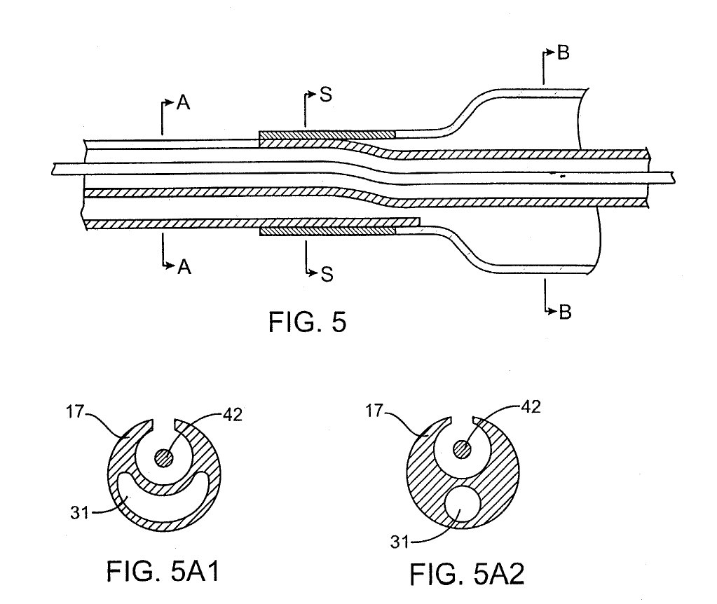

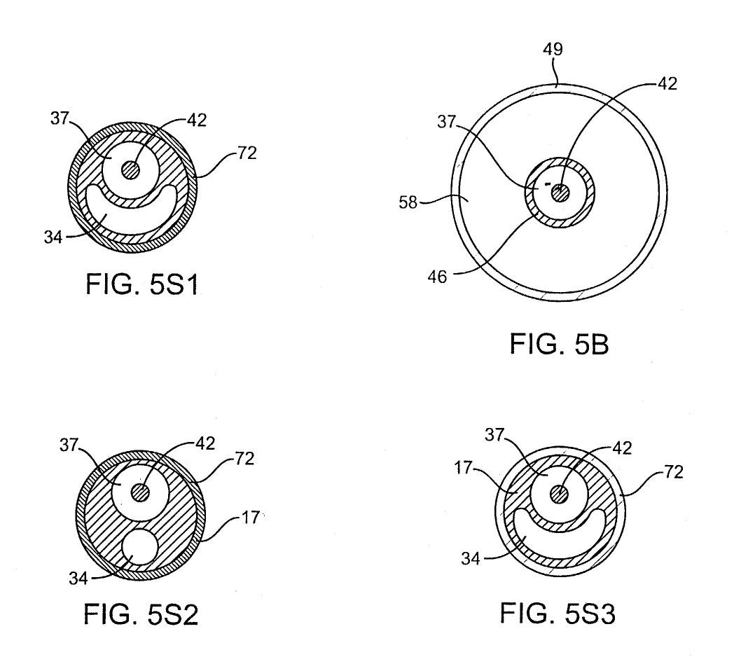

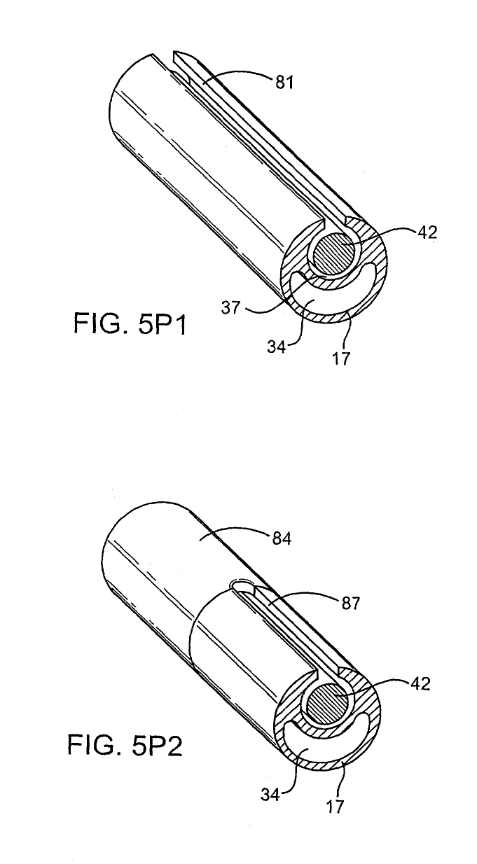

FIGS. 1 through 4 illustrate features of an intravascular catheter assembly 10 embodying features of the present invention and generally including an intravascular catheter 13 comprising a catheter shaft 16 including a flexible tubular shaft 17, proximal 19 and distal ends 22 with a distal tip 23, proximal 25 and distal sections 28, and a lumen 31 extending along at least a portion thereof. At least one lumen such as an inflation lumen 34 extends along at least a portion of the catheter shaft 16 terminating at a point proximal to the distal end 22 of the catheter shaft. In the embodiment shown, the catheter shaft further includes a second lumen 37 for receiving an elongate member 40, such as guidewire 42, tubular body 43 with a lumen 44, or tubular body 43 with the guidewire 42 disposed therein. The proximal end of the catheter shaft, as shown in the exemplary embodiments of FIGS. 2P1-4 may include only the inflation lumen 34, or the inflation lumen as well as the elongate member receiving lumen 37, as shown in FIG. 2P5. The catheter shaft elongate member receiving lumen 37 may be formed, along at least a portion thereof, by a tubular body 46 disposed within the catheter shaft lumen 31 as shown in FIG. 3 and the corresponding transverse cross-sections. Alternatively, the inflation lumen 34 and the elongate member receiving lumen 37 may be formed by a flexible tubular shaft 17′ having a unitary dual lumen construction, as for example by way of extrusion, as shown in FIG. 5. It should be appreciated that the flexible tubular shaft, and/or its lumens may be formed from a single longitudinally extending construction or from a plurality of longitudinally extending and fluidically connected lumens. An expandable balloon 49 is disposed at the catheter shaft distal section 28, having proximal 52 and distal 55 ends and an expandable interior 58 disposed therebetween and in fluid communication with the inflation lumen 34. The distal tip 23 may be tapered for easier tracking along the intravascular path. The distal tip 23 may be formed integrally with the remainder of the catheter shaft 17, or may be formed separately and attached using adhesives, heat fusion, or other techniques. In some instances, the tip 23 may be formed from a particularly soft material to enhance atraumatic introduction of the catheter.

The intravascular catheter 13 of present invention, as shown in FIG. 1 is disposed, in part, within a guiding catheter 61. The catheter assembly 10, as shown includes a luer-type fitting 63 mounted on a proximal extremity 66 of the catheter shaft 16 and is adapted for connection to a syringe or other type of instrument for introducing a radiographic contrast liquid into the catheter shaft 16. For purposes of clarity, it should be appreciated that, not all possible components of the assembly usable with the devices of the present invention for use as a dilatation or stent delivery system or other uses, may have been shown.

In one embodiment, as shown in FIG. 3 a flexible tubular member 69 such as sleeve 72 extends distally from the balloon proximal end 52 and forms a fluid-tight seal with the flexible tubular shaft 17, and provides fluid communication between the inflation lumen 34 and the balloon interior 58 by way of balloon inflation lumen 75. Alternatively, the flexible tubular member 69 may be formed integral with the balloon 49, as shown in FIG. 3S2, or simply be an extended balloon proximal shaft. The flexible tubular shaft 17 may be coextending with the flexible tubular member 69 forming a butt-joint therewith. Alternatively, the flexible tubular member 69 may form an overlap joint with the flexible tubular shaft 17, as shown in FIG. 3S3.

The flexible tubular shaft 17, includes, along at least a portion of its length, access 78, such as longitudinal access 81, for slidably directing the elongate member 40 along at least a portion of the catheter shaft 16, more particularly, within at least a portion of the elongate member receiving lumen 37.

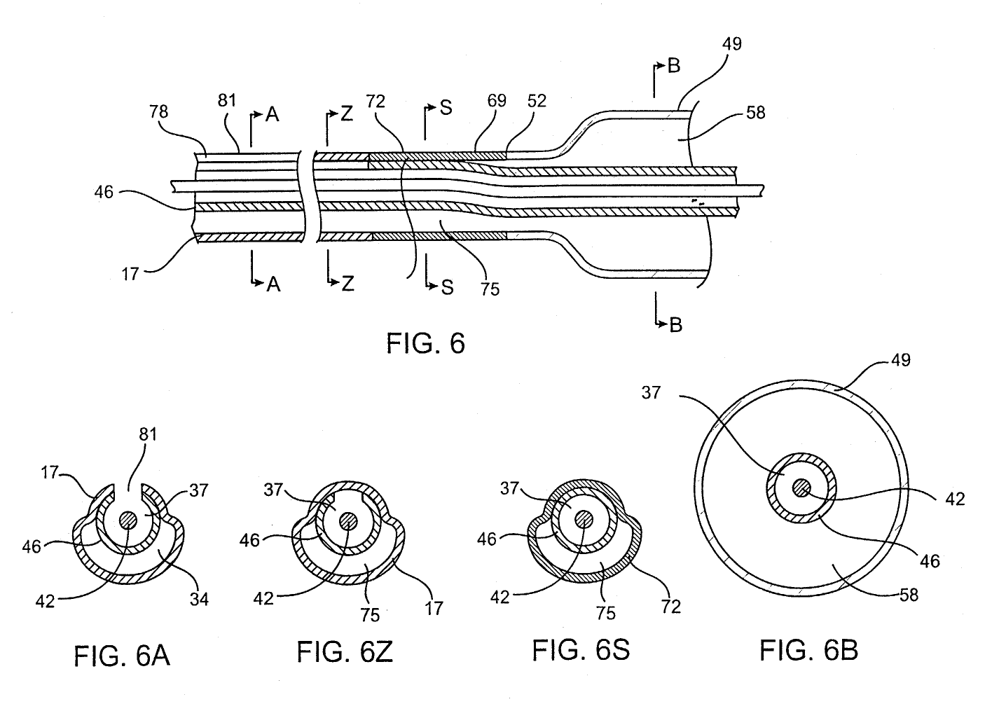

Access 78 may extend distally to a point proximal the proximal end of the flexible tubular member 69, a point proximal to or at the proximal end of the balloon interior, such as a distal point proximal to the flexible tubular member 69, as shown in FIGS. 3 and 6, respectively.

Access 78 may extend proximally to any point along the length of the catheter shaft 17, including but not limited to, the proximal extremity of the flexible tubular shaft 17, a point substantially spaced proximally from the catheter shaft distal end as compared to a distance from the catheter shaft proximal end, a point substantially equidistant from the catheter shaft proximal and distal ends, a point distal of the equidistant point and proximal of the balloon interior, or any other point in between the catheter shaft proximal end and the balloon interior.

FIGS. 3P1-2 and FIGS. P1-2, show a portion of catheters embodying features of the present invention including access 78, with the drawings representing any longitudinal portion of the catheter including the access.

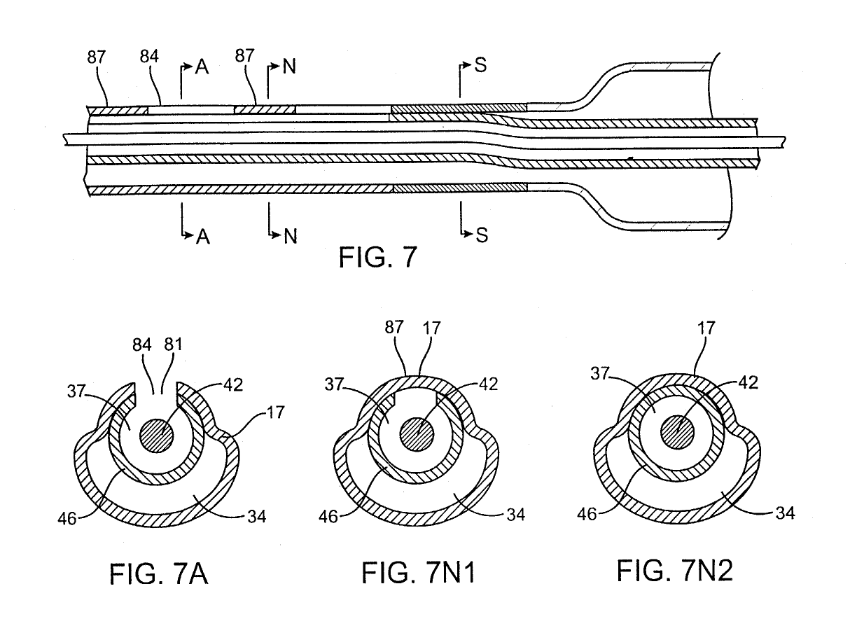

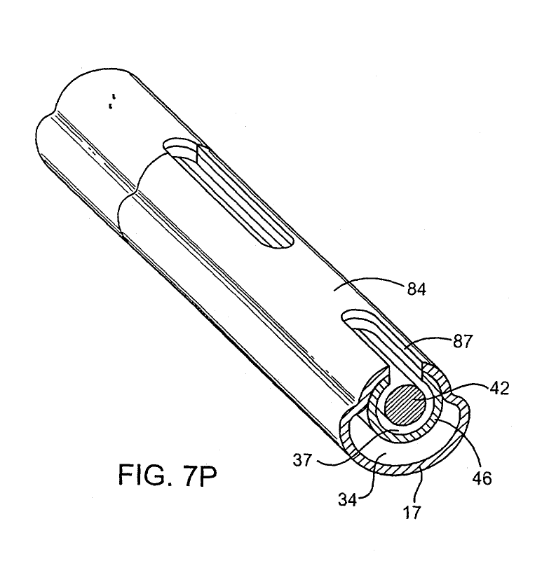

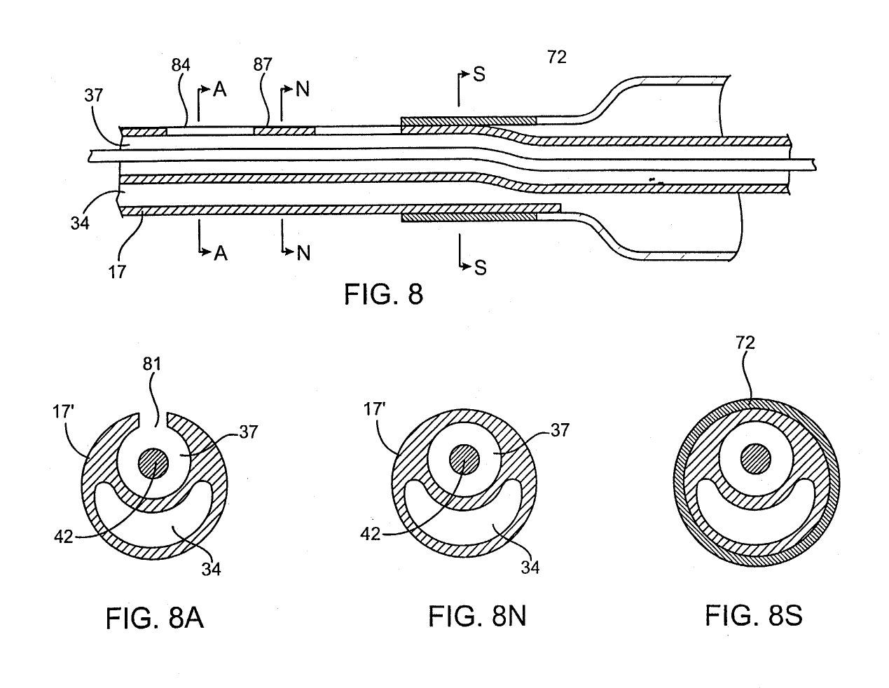

Access 78 may be continuous or formed from a plurality of intermittent access portions 84 longitudinally spaced from one another by non-access portions 87, as shown in FIGS. 7 and 8 and the transverse cross-sections. In one embodiment, access 78 comprises a single continuous access portion 87 along at least the proximal portion thereof and serves as the access area for directing the elongate member including guidewire 42, tubular body 43, or the tubular body housing the guidewire 42. In an embodiment the access 78 comprises a single continuous access portion 87 along the entire length thereof.

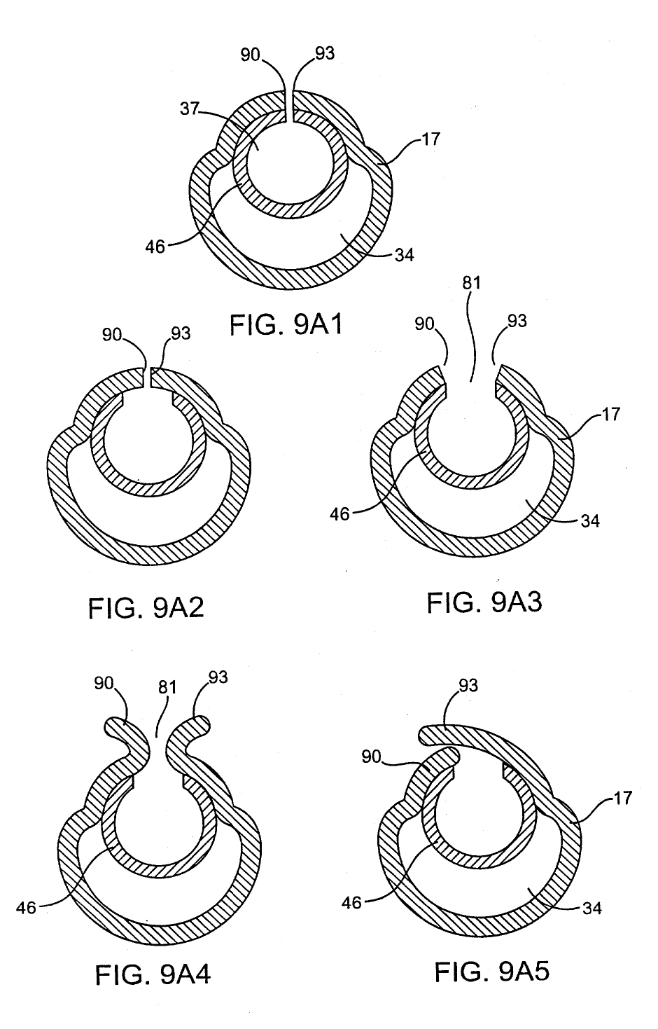

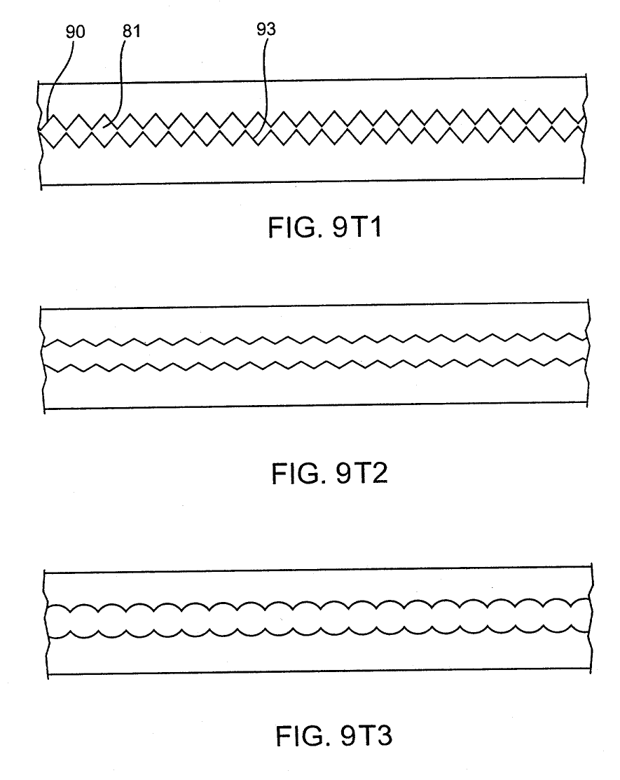

The access 78 may further include transverse ends, as for example longitudinally extending transverse ends, 90 and 93. The transverse ends may overlap, abut, or have an opening formed therebetween. FIGS. 9T1-9T3 and 9A1-9A5, are exemplary embodiments featuring the access and the transverse ends. In an embodiment, the transverse ends 90 and 93 are sufficiently flexible to allow the insertion and/or withdrawal of the elongate member 40 to and from the elongate member receiving lumen 37. In an embodiment, the catheter shaft, along at least the transverse ends is formed of a flexible material having elastic characteristics to enable the retraction of the transverse ends back to a retracted state after the insertion and/or withdrawal of the elongate member to and/or from the elongate member receiving lumen. When the transverse ends form an opening therebetween, the opening is sufficiently large to allow the access of the elongate member (e.g., tubular member or guidewire) to and from the elongate member receiving lumen, yet sufficiently small to minimize the unwanted withdrawal of the elongate member from the elongate member lumen. The opening between the transverse ends may have substantially the same dimension, or may change in the axial direction, as for example increasing or decreasing in the distal direction.

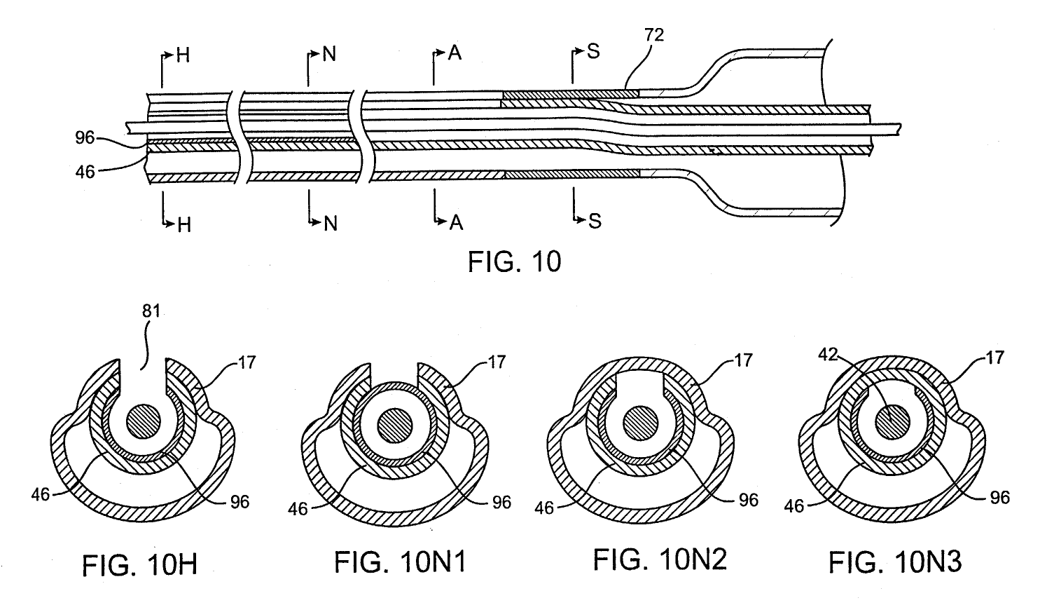

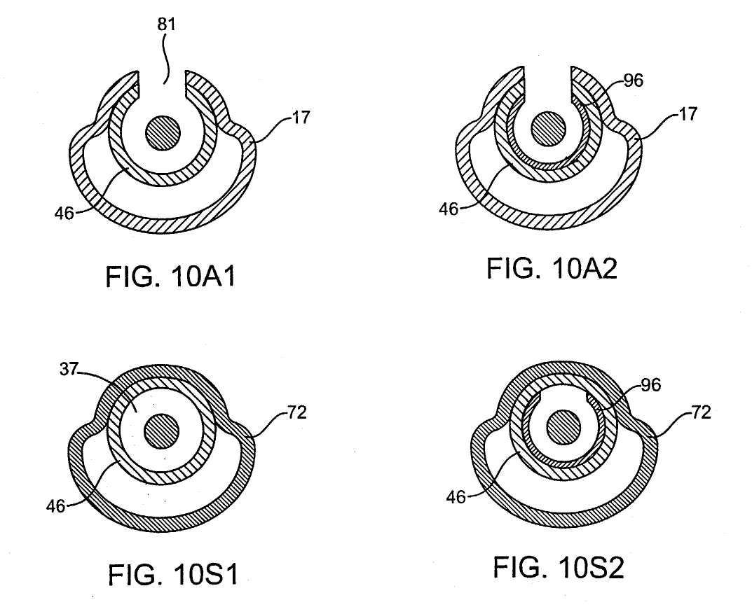

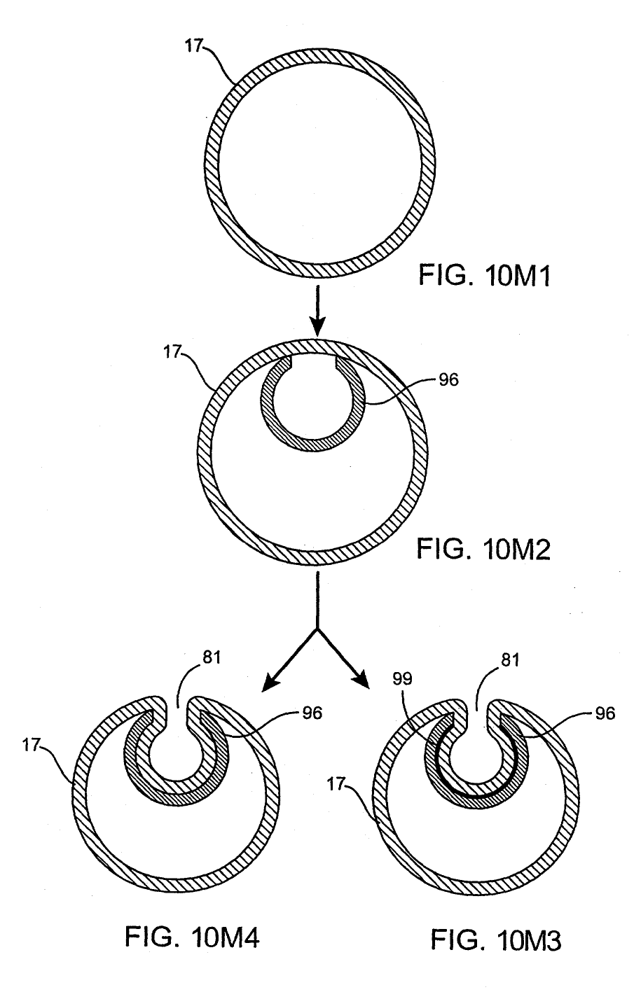

In one embodiment, features of which are shown in FIG. 10, and the corresponding transverse cross-sections, the catheter shaft 16 may further include a stiffening member, including a hypotube 84 extending at least along a portion of its length, more particularly along at least a portion of at least one of its lumens, usually the elongate member receiving lumen 37. The hypotube 84 may extend proximally to the proximal end of the flexible tubular member 69 and distally to a point proximal to the balloon interior, such as a point proximal to or at the flexible tubular member 69. The hypotube 84 may extend proximally to any point along the length of the catheter shaft 17, including but not limited to, the proximal extremity of the flexible tubular shaft 17, a point substantially spaced proximally from the catheter shaft distal end as compared to a distance from the catheter shaft proximal end, a point substantially equidistant from the catheter shaft proximal and distal ends, a point distal of the equidistant point and proximal of the balloon interior, or any other point in between.

The hypotube 84 may be disposed within the flexible tubular shaft 17 by inserting the hypotube into the elongate member receiving lumen 37. The hypotube 84 may be optionally affixed at least along a portion of its length to the surface of the lumen 37. In one embodiment, features of which are shown in FIGS. 10M1-10M4, the hypotube 84 having a longitudinal access along at least a portion of its length may be inserted into lumen 31 of flexible tubular shaft 17, followed by shaping (e.g. crimping) the flexible tubular shaft 17 onto the exterior surface of the hypotube 84, forming an elongate member receiving lumen 37 between transverse ends of the access. The hypotube 84 may be further affixed, at least along a portion thereof, to the flexible tubular shaft 17 by way of suitable means, such as adhesive 99.

The hypotube may be formed from any suitable material such as metals and/or metal alloys, polymers, or combinations thereof; such as stainless steel, nickel-titanium alloy (e.g., Nitinol™ alloy, or combinations thereof. The hypotube may further be coated with coated or contained by a polymer material. Other alternatives include hyptotubes formed from composite material and/or polymers having a different composition profile in the longitudinal direction.

Catheters of the present invention will have dimensions selected to accommodate the particular target location within the intracorporeal body, in particular the vasculature to be treated. Usually the catheter shaft 16 longitudinal dimension ranges from about 10 centimeters (cm) to about 200 cm, usually from about 50 cm to about 200 cm, typically from about 125 cm to about 150 cm for treatment of the coronary vasculature.

The balloon or other expandable structure generally has a length less than that of the sleeve, usually much shorter, typically being in the range from about 4 to about 60 mm, usually from about 10 to about 50 mm; typically from about 20 to 40 mm; balloon having a working length ranging from about 5 to about 40 mm.

The inflation lumen 34 usually has a length similar to that of the catheter shaft less the length of the catheter shaft distal to the termination point of the inflation lumen within the balloon interior, and is usually in the range from about 10 cm to about 150 cm.

The elongate member receiving lumen 37 may generally have a length in the range from about 1 cm to about 200 cm, usually from about 1 cm to about 150 cm, and typically from about 10 cm to about 150 cm.

The elongate member receiving lumen 37 is appropriately sized to accommodate elongate bodies such as guidewires 42 and tubular members 43. The elongate member receiving lumen 37 will usually have an inner diameter ranging from about 0.0145 inches (0.368 millimeters (mm)) to 0.03 inches (0.762 mm), preferably from about 0.016 inches (0.406 mm) to about 0.02 inches (0.512 mm).

Guidewires typically have diameters of about 0.006 inch (0.15 mm) or about 0.008 inch (0.20 mm) to about 0.035 inch (0.89 mm) and the guidewire lumens will typically have diameters in the range from 0.2 mm to 2 mm, usually from or 0.4 mm to 0.6 mm, respectively. In embodiments using a tubular body 43, the outer and inner diameter dimensions of the tubular member will be configured for disposal within the elongate member receiving lumen 37 and receipt of the guidewire 42.

Access 81 may have a longitudinal dimension substantially the same as the flexible tubular shaft 17 less the distance extending from the flexible tubular shaft distal end to the distal end of the access. In one embodiment, access 81 has a longitudinal dimension further reduced at the proximal end, by a distance extending from the flexible tubular shaft 17 proximal end to a substantially equidistant point from the a flexible tubular shaft proximal and distal ends, the equidistant point defining the proximal end of the access; or to a point closer to the flexible tubular shaft distal end than to the flexible tubular shaft proximal.

The portion of the catheter shaft extending between the catheter shaft distal end and the most distal point of the access of the elongate member receiving lumen 37 may have a length ranging from about 3 cm to about 50 cm, usually from about 4 cm to about 40 cm, and typically from about 5 cm to about 25 cm.

Typically, the opening between the access transverse ends is configured to accommodate the directing of the elongate member 40 to and from the elongate member receiving lumen 37, and may range from about 0.01 inches to about 0.1 inches, and usually from about 0.001 inches to about 0.014 inches.

The catheter and the tubular bodies may be formed from polymeric, composite, braided, metallic material, or combinations thereof. Typically, the tubular bodies may be formed as extrusions of polymeric resins. Suitable resins materials include polyamides (nylons), polyimides, polyvinylchloride, PEBAX, PTFE, and the like. Catheter bodies may optionally be reinforced with braids, coils, filaments or other materials in order to enhance the pushability and/or reduce the wall thickness.

In an exemplary method embodying features of the present invention a guiding catheter 61 is inserted into the coronary artery in a conventional manner. The device of the present invention, such as the balloon catheter is prepared for insertion into the guiding catheter 61 in a conventional manner. An elongate member 40 such as the guidewire 42 is then introduced into the catheter 13 by a back loading technique. The proximal extremity of the guidewire 42 is inserted backwardly through the distal tip at the distal end of the catheter through the elongate member receiving lumen 37. The guidewire is advanced rearwardly by holding the distal extremity of the balloon catheter in one hand and advancing the guidewire rearwardly. The guidewire may be advanced toward the proximal end of the catheter either within or outside the elongate member receiving lumen. However, the guidewire is preferably maintained within the elongate member receiving lumen during this proximal advancement. The guidewire is advanced proximally until the distal end of the guidewire with its flexible or floppy tip protrudes at least partially from the distal end of the catheter.

The distal end of the catheter 13 with the flexible tip of the guidewire protruding therefrom is then slid down the guiding catheter 61 previously positioned within the patient. The catheter 13 with the guidewire positioned within the elongate member receiving lumen is grasped between the fingers of a hand and is advanced into the guiding catheter. This procedure is continued until a substantial portion of the catheter is disposed in the guiding catheter. With the catheter housing the guidewire in the catheter elongate member receiving lumen during this distal advancement, improved pushability may be achieved.

In one embodiment, the catheter with the guidewire housed within the catheter elongate member receiving lumen is held stable by the fingers of the hand and is advanced distally until the distal end of the guidewire crosses the stenosis which it is desired to be opened or enlarged and the therapeutic or diagnostic portion such as the balloon 49 is at the desired lesion site. Since the guidewire and the catheter are advanced, at least mostly, together, greater pushability can be obtained in advancing the balloon dilatation catheter across the stenosis. In other words, more force can be applied to the balloon to cause it to cross the stenosis or lesion in case the opening therein is very small.

Alternatively, if desired, the proximal extremity of the guidewire may be disposed outside of the catheter elongate member receiving lumen and a torquer can be attached to the guidewire near the proximal end of the guidewire. The guidewire is then advanced ahead of the catheter until it enters the arterial vessel of the patient. The catheter with the guidewire housed within its elongate member receiving lumen is held stable by the fingers of the hand while the guidewire is being advanced distal of the catheter distal end. The positioning of the guidewire in the desired arterial vessel can be observed under a fluoroscope by using x-ray techniques well known to those skilled in the art. As is well known to those skilled in the art, the torquer can be utilized for rotating the guidewire to facilitate positioning of the flexible tip in the desired arterial vessel so that the distal end of the guidewire can be advanced into the stenosis which it is desired to open or enlarge. As soon as the guidewire is in the desired location, with the exposed proximal extremity of the guidewire held stationary by two fingers of the hand, the catheter is advanced over the guidewire until the therapeutic or diagnostic portion such as the balloon is at the desired lesion. If any difficulty is encountered by the operator conducting the procedure in introducing the catheter so that the balloon resists crossing the lesion or stenosis, the guidewire can be retracted slightly. The person then can observe under the fluoroscope to see that the tip of the guidewire is wiggling in the blood stream indicating that it is free to move in the blood stream. Then the operator can grasp both the guidewire and the catheter in one hand and advance them as a unit so that they can cross the stenosis as a unit.

After the balloon has crossed the stenosis or lesion, the balloon can be inflated in a conventional manner by introducing a radiopaque contrast liquid through the catheter inflation lumen 34. After the inflation has occurred and the desired operation has been performed by enlarging the opening in the stenosis, the catheter can be removed very rapidly by the person performing the procedure by grasping the a proximal extremity of the guidewire outside of the catheter elongate member receiving lumen 37 by two fingers (if a torquer has been used, the torquer may be removed prior to this step).

As the catheter is being retracted proximally out of the guiding catheter, more of the guidewire proximal extremity is removed from the catheter elongate member receiving lumen. If desired, the remainder of the catheter can be removed from the guiding catheter until the distal end of the catheter passes over the proximal end of the guidewire. At this point, if desired, a second catheter (e.g., a second balloon catheter, a second catheter carrying a deployable prosthesis such as a stent) can be loaded onto the guidewire in a rearward direction by introducing the proximal end of the guidewire into the distal tip of the second catheter's elongate member receiving lumen. In an embodiment, the second catheter is a catheter embodying features of the present invention in which event, the second catheter's elongate member receiving lumen may be advanced distally over the guidewire, preferably, with a substantial portion of the guidewire length remaining in the second catheter's elongate member receiving lumen.

The second catheter and the guidewire housed, preferably, substantially within the second catheter's elongate member receiving lumen can then be advanced to the desired location as described before.

It should be appreciated that catheters embodying features of the devices of the present invention may include other types of catheters than balloon dilatation and stent deployment catheters and that these features may be employed in the design and use of other catheters which may require advancement over another elongate member.

Although certain embodiments and methods have been disclosed herein, it will be apparent from the foregoing disclosure to those skilled in the art that variations and modifications of such embodiments and methods may be made without departing from the true spirit and scope of the invention. Therefore, the above description and figures should not be taken as limiting the scope of the invention which is defined by the appended claims.

What is claimed is:

1. An intravascular catheter, comprising:
an elongate catheter shaft having
a proximal end and a distal end with a distal port;
a proximal section including the proximal end, and a distal section including the distal end;
an elongate member receiving lumen having proximal and distal ends and extending between the proximal and distal sections for receiving an elongate member therein; and
an access for directing the elongate member to or from the elongate member receiving lumen.

2. A catheter as in claim 1 wherein the elongate member receiving lumen extends distally from a point substantially at the catheter proximal end.

3. A catheter as in claim 1 wherein the elongate member receiving lumen extends distally from a point distal to the catheter shaft proximal end and substantially closer to the shaft proximal end than to the shaft distal end.

4. A catheter as in claim 1 wherein the elongate member receiving lumen extends distally from a point distal to the catheter shaft proximal end and substantially at equal distance from the shaft proximal and distal ends.

5. A catheter as in claim 1 further including a therapeutic or diagnostic element at the catheter shaft distal section.

6. A catheter as in claim 1 wherein the therapeutic or diagnostic element has proximal and distal ends.

7. A catheter as in claim 1 wherein the diagnostic or therapeutic element is a balloon having proximal and distal ends.

8. A catheter as in claim 7 wherein the elongate member receiving lumen extends distally from a point distal to the catheter shaft proximal end and proximal to the balloon proximal end.

9. A catheter as in claim 1 or wherein the elongate member receiving lumen extends distally to a port at the catheter shaft distal end.

10. A catheter as in claim 1 wherein an access proximal end is coincident with the proximal end of the elongate member receiving lumen.

11. A catheter as in claim 1 wherein an access proximal end is distal to the elongate member receiving lumen proximal end.

12. A catheter as in claim 11 wherein the access proximal end is substantially closer to the shaft proximal end than to the shaft distal end.

13. A catheter as in claim 11 wherein the access proximal end is at equal distance from the shaft proximal and distal ends.

14. A catheter as in claim 1 or 7 wherein the access proximal end is proximal to a balloon proximal end.

15. A catheter as in claim 1 wherein the elongate member receiving lumen extends along substantially the entire length of the catheter shaft.

16. An intravascular catheter for performing a procedure at a location within a patient's coronary artery configured for use with a guiding catheter having proximal and distal ends and an inner lumen extending therein with the guiding catheter distal end configured to be seated within an ostium of the patient's coronary artery and a guiding catheter proximal end extending out of the patient, comprising:
an elongate catheter shaft having
a proximal end and a distal end with a distal port;
a proximal section including the proximal end, and a distal section including the distal end;
an elongate member receiving lumen having proximal and distal ends and extending between the proximal and distal sections for receiving an elongate member therein; and
an access for directing the elongate member to or from the elongate member receiving lumen.

17. A catheter as in claim 16 wherein the elongate member receiving lumen is configured to be spaced sufficiently proximally from the catheter shaft distal end to remain within the guiding catheter during the procedure.

18. A catheter as in claim 16 wherein the elongate member receiving lumen is configured to be spaced sufficiently proximally from the catheter shaft distal end to remain within a substantially straight portion of the guiding catheter during the procedure.

19. A catheter as in claim 16 wherein the elongate member receiving lumen is configured to extend proximally from the guiding catheter proximal end during the procedure.

20. A catheter as in claim 17 or 18 wherein the portion of the elongate member receiving lumen remaining within the guiding catheter includes at least a portion of the access.

21. A catheter as in claim 20 wherein the portion of the elongate member receiving lumen remaining within the guiding catheter during the procedure includes at least a portion of the access.

22. A catheter as in claim 1 wherein the access is configured to at least partially extend distally from the guiding catheter distal end during the procedure.

23. A catheter as in claim 1 wherein the access is configured to at least partially extend proximally from the guiding catheter proximal end during the procedure.

24. An intravascular catheter, comprising:
an elongate catheter shaft having
a proximal end and a distal end with a distal port;
a proximal section including the proximal end, and a distal section including the distal end;
an elongate member receiving lumen having proximal and distal ends and extending between the proximal and distal sections for receiving an elongate member therein; and
an access for directing the elongate member to or from the elongate member receiving lumen and being spaced proximally from the catheter shaft distal end in a range from about 3 to about 50 centimeters.

25. A catheter as in claim 24 wherein the intravascular catheter is a balloon catheter.

26. A catheter as in claim 24 or 25 wherein an access distal end is spaced from the catheter shaft distal end in a range from about 3 to about 50 centimeters.

27. A catheter as in claim 26 wherein the access distal end is spaced from the catheter shaft distal end in a range from about 10 to about 40 centimeters.

28. A catheter as in claim 26 wherein the access distal end is spaced from the catheter shaft distal end in a range from about 15 to about 35 centimeters.

29. A catheter as in claim 26 wherein the access distal end is spaced from the catheter shaft distal end in a range from about 20 to about 30 centimeters.

30. A catheter as in claim 26 wherein the access distal end is spaced about 25 cm from the catheter shaft distal end.

31. A catheter as in claim 24 or 25 wherein the access is formed from a continuous access.

32. A catheter as in claim 24 or 25 wherein the access is formed from a plurality of access portions spaced from one another by non-access portions.

33. A catheter as in claim 24 or 25 wherein the elongate member receiving lumen is in fluid communication with outside of the catheter shaft by way of the access.

34. An intravascular catheter, comprising:
an elongate catheter shaft having
a proximal end and a distal end with a distal port therein;
a proximal section including the proximal end, and a distal section including the distal end;
an elongate member receiving lumen having proximal and distal ends and extending between the proximal and distal ends for receiving an elongate member therein; and
an access for directing the elongate member to or from the elongate member receiving lumen and extending from the elongate member receiving lumen proximal end to a point proximal the elongate member receiving lumen distal end.

35. A catheter as in claim 34 wherein the catheter is a balloon catheter further including an expandable balloon at the catheter shaft distal section.

36. A catheter as in claim 35 wherein the access distal end is a point proximal the expandable balloon.

37. An intravascular balloon catheter for performing a procedure at a location within a patient's coronary artery configured for use with a guiding catheter having proximal and distal ends and an inner lumen extending therein with the guiding catheter distal end configured to be seated within an ostium of the patient's coronary artery and a guiding catheter proximal end extending out of the patient, comprising:
an elongate catheter shaft having
a proximal end and a distal end with a distal port thereat;
a proximal section including the proximal end, and a distal section including the distal end;
an elongate member receiving lumen having proximal and distal ends and extending between the proximal and distal sections for receiving an elongate member therein; and
an access for directing the elongate member to or from the elongate member receiving lumen to the catheter distal port.

38. A catheter as in claim 37 wherein the elongate member comprises a tubular body and a guidewire disposed at least partially therein.

39. A catheter as in claim 38 wherein the tubular body has a longitudinal dimension shorter than a longitudinal dimension of the guidewire.

40. A catheter as in claim 39 wherein the access extends proximally from substantially the catheter shaft proximal end.

41. A catheter as in claim 40 wherein the access extends distally to a point substantially at an equidistant point from the proximal and distal ends of the catheter shaft.

42. A catheter as in claim 37 wherein the elongate member receiving lumen is configured to maintain at least a substantial portion of the elongate member length being proximal the flexible tubular member distal end during the procedure.
